(19) United States
(12) Patent Application Publication
(10) Pub. No.: US 2011/0171628 A1
(43) Pub. Date: Jul. 14, 2011

(54) CERVICAL SCREENING ALGORITHMS

(75) Inventors: Christophorus Joannes Lambertus Maria Meijer, Leiden (NL); Petrus Josephus Ferdinandus Snijders, Amstelveen (NL); Renske Daniëla Maria Steenbergen, Aalsmeer (NL); Daniëlle Anne Marie Heideman, Amsterdam (NL)

(73) Assignee: Vereniging Voor Christelijk Hoger Onderwijs, Wetenschappelijk Onderzoek En Patientenzorg, Amsterdam (NL)

(21) Appl. No.: 12/922,131

(22) PCT Filed: Mar. 13, 2009
(86) PCT No.: PCT/NL2009/050122

§ 371 Date: Dec. 6, 2010

(30) Foreign Application Priority Data
Mar. 13, 2008 (EP) 08152698.0

(51) Int. Cl. C12Q 1/70; C12Q 1/68

(52) U.S. Cl. 435/5; 435/6.11

(57) ABSTRACT

This invention describes new protocols for screening for cervical carcinomas or high-grade premalignant cervical lesions based on combinations of testing for the presence of high-risk HPV, HPV genotyping, marker analysis, and/or cytology. With these protocols the number of women that have to undergo follow-updiagnostic testing and/or clinical examinations will decrease. Further, the number of false positives and false negatives will decrease.

### FIELD OF THE INVENTION

The invention relates to the field of cancer prevention and medical diagnostics; more specific the diagnostics of cancer and precancerous lesions, in particular cervical cancer and its precancerous lesions.

### BACKGROUND OF THE INVENTION

Cancer of the uterine cervix is the second most common cancer in women world-wide and is responsible for approximately 250.000 cancer deaths a year.

Cervical cancer development is characterized by a sequence of premalignant lesions, so called cervical intraepithelial neoplasia (CIN) lesions, which are graded 1 to 3, referring to mild dysplasia (CIN 1), moderate dysplasia (CIN 2) and severe dysplasia/carcinoma in situ (CIN 3). In principle, these premalignant lesions are reversible, although the more severe the lesion, the lower the chance of spontaneous regression. Cervical cancer is considered a preventable disease because the premalignant stages can be detected by exfoliative cytology and treated relatively easily when necessary, with only minor side effects. Cervical screening is aimed to early diagnose the premalignant and treatable cancerous lesions, thereby reducing the mortality of invasive cervical cancer. General medical practice comprises the treatment of all women with morphologically confirmed CIN 2 and CIN 3, in order to prevent the development of cervical cancer.

The problem with current population-based cervical screening programs that use cytological examination (i.e. the Papanicolaou or Pap test) is that this test suffers from a suboptimal sensitivity (at maximum 70%) for cervical carcinoma and closest precursor lesions (i.e. lesions ≧CIN 2/3) and reproducibility, which in practice leads to a substantial number of false negative and false positive test results. Moreover, cytology is not an option for self-sampled cervico-vaginal specimens that can be taken at home, since these are not representative for the cytological status of the cervix (Brink et al., 2006, J. Clin. Microbiol. 44:2518-2523). Therefore, alternative screening tools are under evaluation.

Over the past decades it has been well established that cervical carcinogenesis is initiated by an infection with high-risk human papillomavirus (hrHPV). This causal relationship becomes evident from epidemiological and functional studies (zur Hausen, Nat Rev Cancer 2002; 2:342-350; Bosch et al., J Clin Pathol. 2002; 55: 244-265). HrHPV DNA has been detected in up to 99.7% of cervical squamous cell carcinomas (SCCs) (Walboomers et al., J. Pathol. 1999: 189: 12-19) and at least 94% of cervical adeno- and adenosquamous carcinomas (Zielinski et al., J Pathol 2003: 201: 535-543). Expression of the viral oncogenes E6 and E7, which disturb the p53 and pRb tumor suppressor pathways, respectively, has been shown to be essential for both the onset of oncogenesis and the maintenance of the malignant phenotype. Therefore, testing for hrHPV is an attractive, alternative primary screening tool.

However, consistent with a multistep process of carcinogenesis, additional alterations in the host cell genome are required for progression of an hrHPV infected cell to an invasive carcinoma. In line with multiple events underlying cervical carcinogenesis is the observation that only a small proportion of women infected with hrHPV will develop CIN 2/3 or cervical cancer, and in most women with low grade premalignant cervical lesions the lesions regress spontaneously. Of the women who participate in population based screening about 5-6% have a positive hrHPV test (Bulkmans et al., Int J Cancer 2004, 110:94-101). However, only at maximum 20% of them (1% of the participating women) have ≧CIN 2/3. Therefore, primary screening by hrHPV testing will be accompanied with a substantial number of redundant follow-up procedures and unnecessary anxiety amongst women, unless markers can be applied that allow stratification of hrHPV positive women for risk of ≧CIN 2/3. A major challenge is to reduce the percentage of test positive women to those that have clinically meaningful lesions. One mode is to use cytology as a secondary (so-called triage) test for hrHPV positive women. Still, this leaves a substantial number of hrHPV positive women with normal cytology (3.5% of the women in the screening population), of which 10% have or acquire ≧CIN 3.

In addition, in some circumstances, such as self-sampling, cytology is not an option.

Therefore, there is a need for supplementary or alternative triage tools to stratify hrHPV positive women into those with and without ≧CIN 2/3. Novel insight into cervical carcinogenesis obtained from studies performed in our lab has now yielded a unique set of markers on the basis of which novel screening algorithms can be designed. Marker assays involve tests that analyse over-/under-expression of a set of host cell genes/proteins (i.e., at the level of DNA copy number, mRNA expression or protein expression) and/or hypermethylation of a set of host cell genes and/or the promoter regions thereof (referred to as expression and methylation markers, respectively), either or not supplemented with hrHPV type information. The screening algorithms include primary screening by hrHPV testing and typing, followed by molecular testing without cytology for the triage of hrHPV positive women, or followed by reflex cytology and subsequent further triage of hrHPV positive women with normal cytology by molecular testing. It is obvious for a person skilled in the art that the order of tests is subject to changes without having a major impact on the outcome, e.g. marker analysis could be performed first, either or not followed by hrHPV testing and typing and/or cytology.

### SUMMARY OF THE INVENTION

The inventors now have developed screening protocols, which are unique for the screening of women for cervical cancer and the precursors thereof. They allow, with a high specificity, the identification of women who should be referred for colposcopy because of their high risk of having the precursor CIN2/3.

Thus comprised in the invention is a method for cervical screening of women comprising
a) detection of hrHPV in cervical, cervico-vaginal or vaginal samples (i.e. cells, tissues or fluids thereof);
b) subjecting women positive in a) to marker analysis;
c) referring women positive in b) for colposcopy.
d) keeping women positive in a) for HPV 16, 18, and/or 45 and negative in b) under close surveillance
e) referring women negative in a) or positive in a) for HPV types different from HPV 16, 18, and/or 45 and negative in b) to the next screening round.

Alternatively in said method, women positive in a) for HPV types different from HPV 16, 18, and/or 45 and negative in b) undergo follow-up testing after a time interval of maximally 3 years to minimize the risk of interval high-grade lesions.

Alternatively, all hrHPV positive women are subjected to cytology first, and only women having a cytologically normal smear are subsequently managed on the basis of HPV type and marker test results. Thus, comprised in the invention is a method for cervical screening of women comprising
a) detection of hrHPV in cervical, cervico-vaginal or vaginal samples (i.e. cells, tissues or fluids thereof);
b) subjecting women positive in a) to cytology;
c) referring women with abnormal cytology for colposcopy;
d) subjecting women with normal cytology to marker analysis;
e) referring women positive in d) for colposcopy; keeping women with normal cytology positive in a) for HPV 16, 18, 31, 33, and/or 45 and negative in b) under close surveillance
g) referring women negative in a) and those with normal cytology positive in a) for HPV types different from HPV 16, 18, and/or 45 and negative in d) to the next screening round

Alternatively in said method, women with normal cytology positive in a) for HPV types different from HPV 16, 18, and/or 45 and negative in d) undergo follow-up testing after a time interval of at maximum 3 years to minimize the risk of interval high-grade lesions.

Alternatively, women are subjected to marker analysis first, and only women having a marker-negative test result are subsequently subjected to hrHPV testing and typing. Thus, comprised in the invention is a method for cervical screening of women comprising:
a) subjecting women to marker analysis;
b) referring women positive in a) to colposcopy;
c) subjecting women negative in a) to hrHPV testing and typing;
d) keeping women positive in c) for HPV 16, 18, anchor 45 under close surveillance
e) referring women negative in c) and those positive in c) for HPV types different from HPV 16, 18, and/or 45 to the next screening round

Alternatively in said method, women positive in c) for HPV types different from HPV 16, 18, anchor 45 undergo follow-up testing after a time interval of at maximum 3 years to minimize the risk of interval high-grade lesions.

Alternatively, women are subjected to marker analysis first, and only women having a marker-negative test result are subsequently subjected to cytology. Thus, comprised in the invention is a method for cervical screening of women comprising:
a) subjecting women to marker analysis;
b) referring women positive in a) to colposcopy;
c) subjecting women negative in a) to cytology;
d) referring women with abnormal cytology to colposcopy
e) referring women with normal cytology to the next screening round

Alternatively in said method, women negative in c) undergo HPV testing and typing. Women negative for HPV will be referred to the next screening round. Women positive for HPV types HPV 16, 18, and/or 45 will be kept under close surveillance and women positive for HPV types different from HPV 16, 18, and/or 45 will be referred to the next screening round or, alternatively, undergo follow-up testing after a time interval of at maximum 3 years to minimize the risk of interval high-grade lesions.

Additionally, in the above methods, women positive for HPV types 31 and 33 are diagnosed and treated similar to women positive for HPV types 16, 18 and/or 45.

Alternatively, women are subjected to marker analysis only, and women having a marker-positive test result are referred for colposcopy. Thus, comprised in the invention is a method for cervical screening of women comprising:
a) subjecting women to marker analysis;
b) referring women positive in a) to colposcopy;
c) subjecting women negative in a) to the next screening round

In all methods marker analysis involves testing with a marker panel consisting of expression and/or methylation markers, wherein said expression markers are selected from the markers given in Table 1 of the European patent application 07114580, e.g. from the group of ATP2C1 (Genbank ID: NM_{—}014382), SLC25A36 (Genbank ID: NM_{—}018155), DTX3L (Genbank ID: AK025135), CCDC14 (Genbank ID: AL122079), FLJ21291 (Genbank ID: AK024944), ITGAV (Genbank ID: NM_{—}002210), PIK3R4 (Genbank ID: Y08991), and MAL (Genbank ID: NM_{—}022438), and said methylation markers are selected from CADM1 (previously named TSLC1; Genbank ID NM_{—}014333; see patent WO 2004/087962 and MAL (Genbank ID: NM_{—}022438), respectively. A preferred embodiment according to the present invention uses a combination of methylation analysis of CADM1 (Genbank ID NM_{—}014333) and MAL (Genbank ID: NM_{—}022438). Alternatively, said methylation marker involves MAL (Genbank ID: NM_{—}022438). Preferably, the women testing positive for said marker analysis, i.e. abnormal expression of expression markers or methylation of one or more methylation marker genes, are referred for colposcopy.

The overall result of the invention is a method to identify those women that are at highest risk of having or developing high-grade precursor lesions of cervical cancer by performing any of the above described methods and concomitantly to decrease the number of women subjected to redundant or excessive follow-up or colposcopy.

### LEGENDS TO THE FIGURES

FIGS. 1-5 disclose the alternative screening protocols according to the methods of the invention.

### DETAILED DESCRIPTION OF THE INVENTION

Testing for cervical cancer is nowadays often done in large scale population-based primary screening settings, which are based on a standard cytological test (the PAP-test). In the meantime, several molecular biological test assays have become available, such as a PCR assay for detection and typing of HPV (human papillomavirus), and marker analysis, using (gene) expression markers and/or methylation markers. Although all of these methods may indicate (early) stages of cervical cancer and high-grade precursor stages thereof, the final determination of this disease can only be done by colposcopy-directed biopsy. However, this is a labour-intensive, expensive screening method, which is also undesirable from a patient's point of view, especially for the both physical and, more important, psychological well-being of the women subjected to such an invasive diagnostic method.

Now, according to the invention several screening protocols have been developed, which minimise the number of women that would undergo redundant colposcopy. Next to this, the methods of the invention also are reliable in that the number of false-positive and/or false-negative results is much lower than obtained with conventional methods. Further, the methods of the invention enable a stepwise diagnostic screening, which implies that in every step only those patients are involved which have scored (partly) positive in previous steps. This automatically implies that the number of patients to be tested in subsequent steps of the method decreases, which is beneficial in relation to the amount of tests and the economic consequences thereof. Further, it is also beneficial for the patients: because of the lower number of false-positive and/or false-negative results, a more reliable diagnosis can be given, preventing psychological pressure to women who, in the end, appear to be healthy, and avoiding shock-effects in women who after a first ‘favourable’ prognosis later appear to have developed the disease.

In all embodiments of the present invention marker analysis is one of the steps for diagnosing the presence of (precursor lesions of) cervical cancer. Marker analysis can involve the analysis of the expression of several genes/proteins or an analysis of the methylation state of (the promoter region of) several genes in a cervical scrape. Down-regulation and/or over-expression of a series of host-cell genes accompany HPV-mediated carcinogenesis of cervical cells. As is detailed in the experimental part, the present inventors have found a series of genes given in Table 1 of the European patent application 07114580 (among which ATP2C1 (Genbank ID: NM_{—}014382), SLC25A36 (Genbank ID: NM_{—}018155), DTX3L (Genbank ID: AK025135), CCDC14 (Genbank ID: AL122079), FLJ21291 (Genbank ID: AK024944), ITGAV (Genbank ID: NM_{—}002210), PIK3R4 (Genbank ID: Y08991) that is highly expressed in and excellently correlated with cervical carcinoma and/or high-grade CIN lesions, and one gene (i.e., MAL (Genbank ID: NM_{—}022438)) that is down-regulated in these cases. Measuring the overexpression, or down-regulation of the above mentioned genes can be performed using standard methodology, such as real time PCR, either or not using microfluid array platforms, allowing simultaneous detection of multiple targets in one sample using limited amounts of input material. It is also possible to detect over-expression or down-regulation at the protein level, by measuring the concentration of the proteins encoded by the above-mentioned genes, e.g. by immuno- and flow-cytometry assays.

Next to over-expression or down-regulation, promoter hypermethylation is often found in tumor developing tissues, including cervical tissue. The present inventors have established earlier that silencing of TSLC1 (tumor suppressor in lung cancer 1), nowadays renamed as CADM1 (cell adhesion molecule 1), also known as IGSF4, NECL2 or SYNCAM, and located at chromosome 11q23.2 (Genbank ID NM_{—}014333)) is a frequent event in cervical cancer cell lines (see Steenbergen et al., JNCI: 2004, 96:294-305 and WO 2004/087962). In vitro studies revealed a functional involvement of CADM1 inactivation in both anchorage independent growth and tumorigenicity of cervical cancer cells, whereas immortality and proliferation were not affected. This suggests that inactivation of CADM1 might play a crucial role in premalignant lesions to become invasive. Recent comprehensive bisulfite sequencing analysis and extensive methylation specific PCR (MSP) analysis of the promoter region of CADM1 revealed that particularly dense methylation is pivotal for gene silencing resulting in decreased protein expression and the phenotypic consequence thereof. In addition, dense methylation (defined as a positive MSP signal in ≧2 of 3 promoter regions analysed) was significantly associated with ≧CIN3 lesions. Thus, a methylation status of the CADM1 promoter provides a clinically very valuable methylation marker.

Methods to determine hypermethylation (especially for CADM1, Genbank ID NM_{—}014333) are given in WO 2004/087962 and alternative methods are known to a person skilled in the art.

Preferred algorithms according to the invention include a test to determine the presence of mucosal hrHPVs (high-risk human papillomaviruses). Papillomaviruses can in general be classified according to tissue tropism. Although all known human papillomaviruses strictly infect cells of epithelial origin (keratinocytes) and induce ‘epithelial proliferations’ they can be grouped according to their tissue tropism into either mucosal or cutaneous HPV types. The mucosal types (e.g. HPV6 and HPV16), include those HPVs that infect mucosa of the genital and respiratory tracts. The cutaneous types (e.g. HPV1 and HPV8) include those HPVs that infect generally the external skin, including those that cause cutaneous warts and those that cause skin carcinomas in patients suffering from epidermodysplasia verruciformis.

Additionally, mucosal HPVs are broadly classified into low-risk and high-risk types, based on their ability to lead to benign epithelial proliferation or to induce malignant changes in infected cells, respectively. Low risk HPV types such as 6, 11, and 32 are primarily associated with benign lesions or common warts while the high risk types, such as 16, 18, 31, 33, 35, 39, 45, 51, 52, 56, 58, and 59 are primarily associated with premalignant and malignant epithelial lesions. These high-risk types of HPV cause growths that are usually flat and nearly invisible, as compared with the warts caused by low-risk types, e.g. HPV-6 and HPV-11.

Nowadays, based on large epidemiological case-control studies, at least 12 mucosal HPV types were classified as high-risk (carcinogenic) to human beings (i.e. HPV16, -18, -31, -33, -35, -39, -45, -51, -52, -56, -58, and -59). Debate is ongoing about a series of possibly, or candidate high-risk types comprising HPV26, -53, -66, -67, -68, -73, and -82. Twelve HPV types are indicated as low-risk (HPV6, -11, -40, -42, -43, -44, -54, 61, -70, -72, -81, and CP6108), although -6 and -11 are also suggested as possibly carcinogenic to human beings.

The HPV tests that are currently most widely applied are based on detection of the group of hrHPV types as a pool, either or not followed by genotyping. A method to determine hrHPV presence is given in PCT/NL2007/050526.

In some of the embodiments of the present invention, patients who have been found positive in the hrHPV test (where “positive” means that the hc2 reaction did provide DNA/RNA hybrids or the PCR reaction amplified fragments with the used primers, and which thus means that one or more of the above-indicated hrHPVs is present in the sample) are subsequently, or previously, subjected to cytological examination and/or marker evaluation.

The cytological examination can be performed on either classic smears or liquid based cytology preparations. Together with a positive hrHPV test result a positive cytology result (i.e. score of borderline dyskaryosis or worse) points to a high likelihood of high-grade premalignant lesions or cervical cancer and consequently these women should be referred for colposcopy. However, absence of cytological abnormalities does not exclude the presence of high-grade premalignant lesions, especially in case of a positive marker test or in case of the presence of HPV 16, 18, and/or 45 even when the marker test is negative. Therefore, HPV genotyping, marker gene information or the combination thereof provide an ideal triage tool for cytology negative women that are hrHPV positive. For HPV 31 and 33 there seems to be an increased risk for premalignant lesions, but there appears to be a decreased risk that cancer would develop from said premalignant lesions. Thus, testing for HPV 16, 18 and/or 45 is required for the methods of the invention, while testing for HPV 31 and 33 can be optional.

The marker evaluation can involve the analysis of the expression of several genes/proteins and/or an analysis of the methylation state of (the promoter region of) several genes in a cervical scrape as is detailed in the experimental part. A positive marker result points to a high likelihood of high-grade premalignant lesions or cervical cancer and consequently these women should be referred for colposcopy.

In the invention HPV typing information is used to complement marker analysis. By studying large cohorts involving more than 65,000 women, involving both women participating in population-based cervical screening and non-responder women that were offered a self-sampling for hrHPV testing, we found that HPV types 16, 18, (31, 33), and/or 45 confer a significantly increased risk of cervical cancer and high-grade premalignant lesions. Notably, in hrHPV positive women with normal cytology, having single infections with hrHPV types different from particularly HPV 16, 18, 31, and/or 33≧CIN2 lesions were extremely rare. This suggests that to a certain extent the cytological manifestation of ≧CIN2 lesions is type-dependent. Similarly, we collected evidence that the far majority of women with ≧CIN 2 lesions who were CADM1 (Genbank ID NM_{—}014333) and MAL (Genbank ID: NM_{—}022438) methylation marker negative contained HPV type 16, 18 and/or 31.

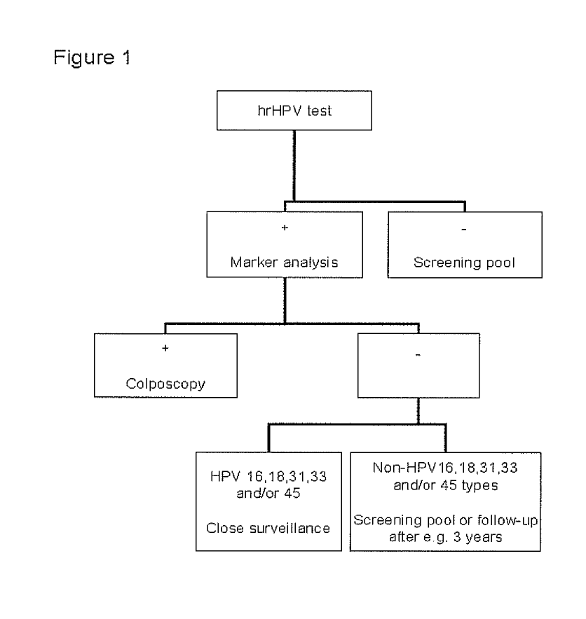

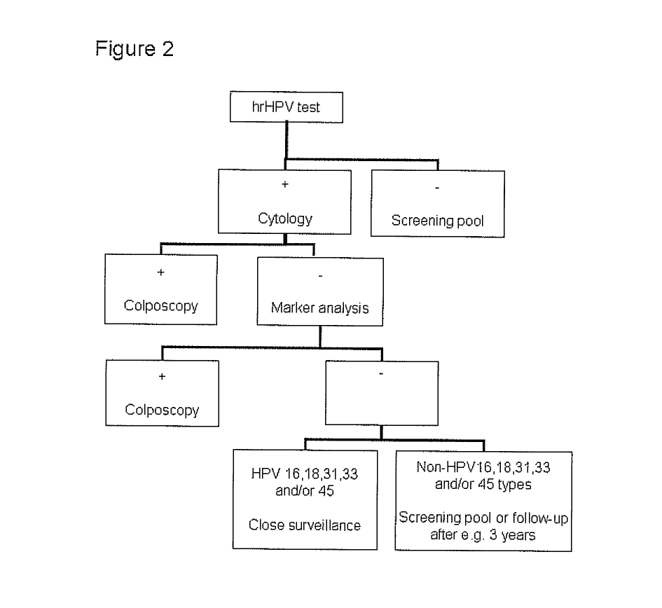

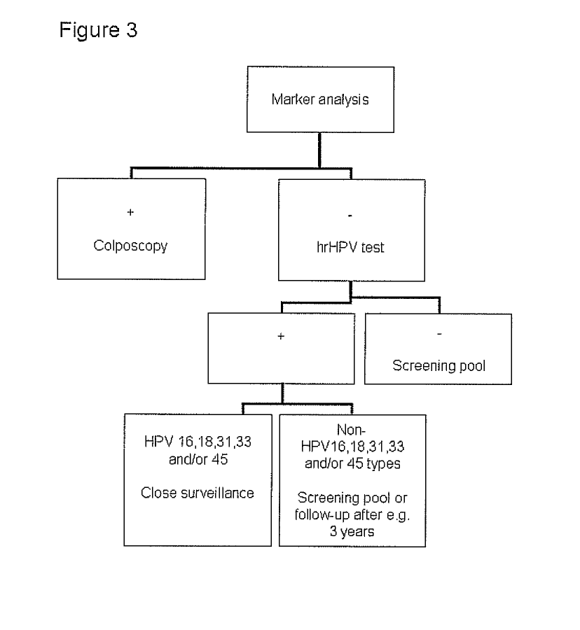

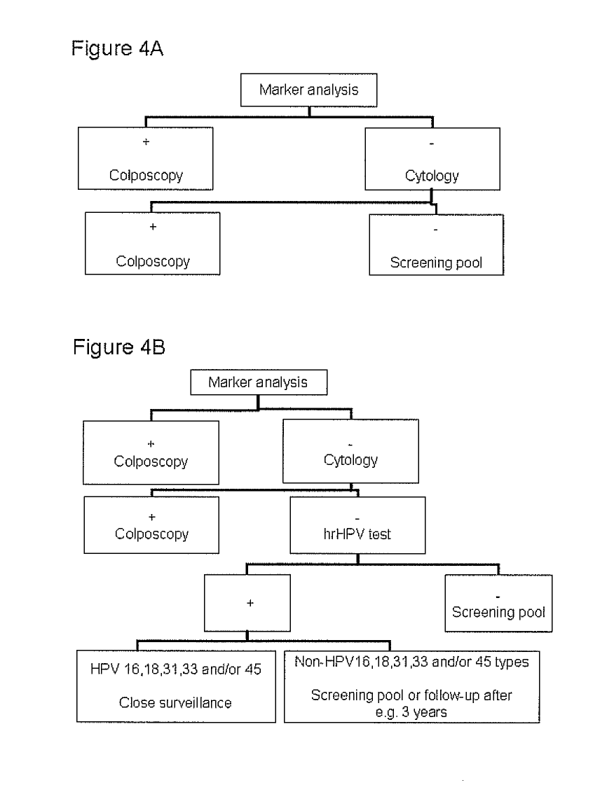

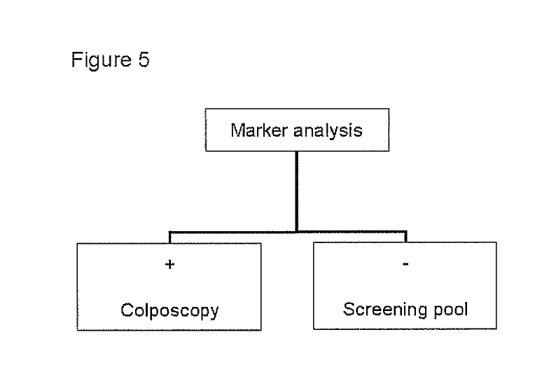

In the invention it is indicated which further steps need to be taken after the outcome of the test is known (see also FIGS. 1-5). One of the algorithm outcomes is referring the woman back to the screening pool (i.e., to the next screening round). This basically means that currently there is no or a negligible risk of having or developing cervical carcinoma within the screening interval. This means that the woman in question can normally participate within the population-based screening at the intervals at which these are held, which is currently at maximum 5 years.

Another possible algorithm outcome is that the particular woman should either be held in close surveillance or undergo follow-up testing after a shorter time interval than the next screening round. In practice the first alternative means examinations on repeat cervical scrapings taken 6 to 12 months later, whereas the second alternative points to examinations on repeat cervical scrapings taken at maximum 3 years later. Depending on the test results on these repeat smears the women will either be referred to the next screening round or for colposcopy.

Lastly, the worst algorithm outcome, i.e. a very high likelihood of ≧CIN 2/3, means that the women will be referred for colposcopy. During colposcopic evaluation of the cervix, most likely biopsies will be taken for close microscopical observation. All women with histologically confirmed CIN 2/3 or worse, are treated according to standard protocols in order to prevent the development of (invasive) cervical cancer.

Thus, the invention provides several alternative algorithms for an efficient and reliable screening of women for risk of having or developing ≧CIN 2/3. The proposed scannings can be performed on a large-scale basis and by the multi-stage nature of the scanning protocols unnecessary testing is prevented, by which the present invention also provides for an economical spending of resources and personnel. Finally, the women who are tested are not subjected to unnecessary testing and further, by the decrease in the number of false positives, the methods of the invention will also decrease the psychological effects of such false positive outcomes.

### EXAMPLES

### 1. HPV Testing

We initiated a prospective, randomized trial to evaluate the effectiveness of hrHPV testing as an adjunct screening tool in a large scale population-based primary screening setting. This POBASCAM (Population based screening Amsterdam) trial compares the yield of ≧CIN 2/3 among 44,102 women in a primary screening program by either cytology testing alone (control arm) or cytology and HPV testing (intervention arm), using a GP5+/6+PCR enzyme immunoassay. Baseline data have been published (Bulkmans et al., Int J Cancer 2004, 110:94-101) and completion of 5 years follow-up of all women is expected in 2008. Interim analyses revealed that the sensitivity and negative predictive value of HPV testing for ≧CIN 2/3 is clearly superior to that of cytology. Data from about 18,000 women who reached already the second screening round (after 5 years) showed that the total number of ≧CIN 2/3 lesions in both arms that accumulated till and including the next screening round was almost the same (Bulkmans et al. Lancet: 2007, 370:1764-1772). However, 73% of the ≧CIN 3 lesions in the intervention arm versus only 47% of those in the control arm were detected prior to the next screening round. As a consequence a reduction of 47% of ≧CIN 2/3 lesions in the next screening round was obtained by adjunct HPV testing. These data indicate that the extra ≧CIN 2/3 lesions detected by adjunct hrHPV testing do not regress and are therefore clinically relevant, indicating that HPV testing does lead to earlier detection of clinically relevant lesions and does not result in an overdiagnosis of CIN 2/3 lesions, which, when left undetected, would regress spontaneously. Moreover, we found that there exist HPV-type specific differences in risk of ≧CIN 2/3. In fact, hrHPV-positive women with types different from type 16, 18, 31, 33 and/or 45 had a relatively low risk of high-grade CIN. In part, this reflects lower clearance rates of these types. To illustrate the importance of types 16, 18, 31, 33 and/or 45 among women with baseline normal cytology, HPV16/18-positive women showed an increased risk of high-grade CIN, even after multiple (n=3) normal smears. This seems to warrant more aggressive management (i.e. more intense follow-up) of HPV16/18-positive women with normal cytology compared to those containing other hrHPV types. In fact, in women with hrHPV positive normal smears having single non-HPV 16, 18, 31, 33 and 35 hrHPV infections no ≧CIN 3 was detected, suggesting that clinically relevant infections with these types are almost exclusively accompanied with cytological abnormalities.

Given these data screening scenarios can be considered with HPV typing information included, particularly to stratify hrHPV positive women who display no detectable pheno- (i.e. cytological) or molecular (i.e. (epi)genetical) alterations in their cervical scrapings.

### 2. Differentially Expressed Genes in Cervical Cancer and HPV-Transformed Epithelial Cells

HPV-mediated carcinogenesis is characterized by both down-regulation and over-expression of a series of host-cell genes. A number of these genes were discovered in our own setting by microarray expression analysis, either or not following an integrated approach of combined microarray expression and microarray CGH (maCGH) analysis. We reasoned that genomic profiling might yield chromosomal signatures representing cervical SCCs and advanced CIN lesions in a highly sensitive and specific manner and that novel markers may be deduced from this approach. The integrated approach allowed the identification of differentially expressed genes that reside at chromosomal locations that show recurrent gains or losses in cervical carcinomas and precancerous lesions. Since non-random chromosomal aberrations are likely to represent crucial genetic events in cervical cancer, genes at these loci showing altered expression may be relevant for the carcinogenic process and therefore have particularly potential as candidate markers.

MaCGH using 5K BAC arrays displayed particularly non-random gains at 1q12-32, 3q25-29, and 20q11-13, found in 78% (1q) to 100% (3q) of the cervical SCCs. Micro array expression analysis of the same set of tumors yielded 24 up-regulated genes, the most significant including ITGAV (Genbank ID: NM_{—}002210, gene location: 2q32.1), and SYCP2 (Genbank ID: NM_{—}014258, gene location: 20q13.33), and 15 down-regulated genes, including MAL (Genbank ID: NM_{—}022438, gene location: 2q11.1. In addition, an integration of genome-wide chromosomal and transcriptional analysis revealed a series of genes (including FLJ21291 (Genbank ID: AK024944, gene location: 1q32.1), DTX3L (Genbank ID: AK025135, gene location: 3q21.1), CCDC14 (Genbank ID: AL122079, gene location: 3q21.1), PIK3R4 (Genbank ID: Y08991, gene location: 3q21.3), ATP2C1 (Genbank ID: NM_{—}014382, gene location: 3q21.3), SLC25A36 (Genbank ID: NM_{—}018155, gene location: 3q23), and ITGAV (Genbank ID: NM_{—}002210, gene location: 2q32.1)) with an increased expression that are mostly located within the regions of non-random gains (see also Tables 1 and 2 of the European patent application 07114580). Interestingly, a more than 2-fold overexpression of at least one of the following genes DTX3L (Genbank ID: AK025135), CCDC14 (Genbank ID: AL122079), FLJ21291 (Genbank ID: AK024944) and PIK3R4 (Genbank ID: Y08991) was found in all cervical carcinomas examined, indicating that a marker panel consisting of these 4 genes may have a 100% sensitivity for cervical carcinoma.

Cross-sectional maCGH analysis of 46 high-grade CIN lesions revealed chromosomal signatures ranging from normal or nearly normal (<5 aberrations; about 50% of cases) to alterations at >10 regions (30% of cases). Interestingly, the signatures of the latter were similar to those of SCC (including the aforementioned gains) and these lesions clustered together with SCCs.

Quantitative RT-PCR analysis of RNA isolated from microdissected normal cervical epithelial samples, microdissected high-grade CIN lesions and SCCs revealed significant overexpression of ITGAV (Genbank ID: NM_{—}002210), ATP2C1 (Genbank ID: NM_{—}014382), SLC25A36 (Genbank ID: NM_{—}018155) and PIK3R4 (Genbank ID: Y08991) in high-grade CIN lesions and carcinomas compared to the normal cervical control samples. Significant overexpression of DTX3L (Genbank ID: AK025135) was restricted to SCCs. MAL (Genbank ID: NM_{—}022438) was found to be significantly down-regulated in both high-grade CIN lesions and SCCs compared to normal controls.

Our data do not only indicate that a valuable marker panel can be composed by combining expression analysis of a subset of host cell genes but also that analysis of DNA copy number gains at the locations with common gains in cervical carcinomas (i.e. 1q, 3q and 20q) can have diagnostic and prognostic power as well for cervical cancer and high-grade precursor lesions.

### 3. Genes with Promoter Hypermethylation

Also down-regulation of a subset of genes is relevant for cervical carcinogenesis. One common mode of gene silencing involves hypermethylation of the promoter region. Molecular markers based on DNA hyper-methylation, also referred to as methylation markers, are of interest since DNA hyper-methylation can be easily detected in cervical scrapes using sensitive PCR based methods like methylation specific PCR (MSP). Moreover, positive MSP results in cervical smears were found to represent the methylation status of respective genes in corresponding biopsies (Feng et al., JNCI: 2005, 282, 273-297).

Our recent studies on HPV-immortalized cell lines, cervical cancer cell lines and cervical samples have yielded a series of candidate methylation markers that can be of value in cervical screening algorithms.

One of the first methylation markers we studied involved the CADM1 gene (Genbank ID NM_{—}014333). We initially showed that inactivation of the CADM1 gene (Genbank ID NM_{—}014333) by promoter hyper-methylation is essential for the maintenance of anchorage and tumorigenic phenotypes of HPV-transformed cells (Steenbergen et al., JNCI: 2004, 96: 294-305; WO 2004/087962). Primarily as a result of promoter methylation this gene was silenced in 91% (9/10) of cervical cancer cell lines. Subsequent comprehensive methylation analysis of the CADM1 promoter indicated that both frequency and density of CADM1 promoter hyper-methylation increases proportional to the severity of (pre)neoplastic cervical disease. Moreover, dense hyper-methylation (i.e. hyper-methylation of at least two of three promoter regions analysed by MSP) was correlated with reduced protein expression of CADM1 (Genbank ID NM_{—}014333) as determined by immunohistochemistry. Dense hypermethylation assessed increased from 5% in normal cervices, to 30% in CIN 3 lesions and 83% in cervical SCC. However, dense CADM1 methylation was more specific for cervical SCC since its frequency was significantly higher in SCC compared to adenocarcinoma (83% vs 23%; p=0.002). In our search for complementary methylation markers that also allow detection of cervical adenocarcinoma and its precursors further microarray expression and CGH studies were performed. From these studies the MAL (Genbank ID: NM_{—}022438) gene was identified as one of the most significantly down-regulated genes in cervical carcinomas compared with normal epithelial control samples. The fact that MAL (Genbank ID: NM_{—}022438) is located at 2q11-13, a chromosomal region at which we did not find recurrent chromosomal deletions in cervical cancer, prompted us to search for a potential epigenetic disregulation of transcription. Treatment of cervical cancer cell lines and HPV-immortalized cell lines with a methylation inhibitor resulted in a strong upregulation of MAL (Genbank ID: NM_{—}022438) mRNA expression. Promoter methylation analysis subsequently confirmed that the MAL promoter region was methylated in these cell lines

In addition, functional analysis revealed that MAL not only has anti-proliferative properties but also inhibits cellular migration and anchorage independent growth of SiHa cells. Hence MAL (Genbank ID: NM_{—}022438) is also functionally involved in cervical cancer development. Next, we evaluated hyper-methylation of MAL (Genbank ID: NM_{—}022438) in clinical materials using quantitative MSP analysis for two regions within the promoter of MAL (Genbank ID: NM_{—}022438). Methylation of one or both regions was found in 4.6% (1/22) of normal cervical samples, 20. % (8/40) of low-grade CIN lesions, 82% (45/58) of high grade CIN lesions and 99% (93/94) of SCCs, and 100% (24/24) of adenocarcinomas.

By combining methylation analysis of these two promoter regions of MAL (Genbank ID: NM_{—}022438) with one newly selected promoter region of CADM1 (Genbank ID NM_{—}014333), the number of methylation-positive high-grade CIN lesions increased to 91% (51/58) (positively was scored in case of a positive result for at least one of these regions). Conversely, by adding analysis of this CADM1 region positively in normal cervices, low-grade CIN lesions and cervical carcinomas was not influenced. Adding methylation data of other genes did not markedly increase these sensitivity figures. We therefore concluded that this combination provides an optimal marker panel for ≧CIN 2/3.

### 4. Marker Gene Analysis of Cervical Scrapes

Methylation analysis was performed on a large series of cervical scrapes of hrHPV GP5+/6+-PCR positive women participating in population-based cervical screening in which ≧CIN 2 was diagnosed within 18 months of follow-up. These included women with abnormal cytology (i.e. borderline dyskaryosis or worse) and normal cytology at baseline, the latter of which were discovered by a positive hrHPV test solely. In addition, hrHPV positive control women with normal cytology and CIN 1 or better within an 18-month follow-up period were included. Baseline cervical scrapes of these women were subjected to various methylation markers including that for above-mentioned CADM1 (Genbank ID NM_{—}014333) region, and two MAL (Genbank ID: NM_{—}022438) regions using quantitative MSP.

Methylation at one or both MAL (Genbank ID: NM_{—}022438) regions varied from 31% in hrHPV positive control women with normal cytology to 65% and 84% in women with ≧CIN 2 having normal and abnormal cytology at baseline, respectively. By combining the latter two groups MAL methylation was found in 79% of women with ≧CIN 2. By adding CADM1 (Genbank ID NM_{—}014333) methylation results, 5% more ≧CIN 2 lesions were detected in women with abnormal cytology, resulting in an overall ≧CIN 2 detection rate of 83%. These data indicate that by combining both MAL (Genbank ID: NM_{—}022438) and CADM1 (Genbank ID NM_{—}014333) methylation markers a high sensitivity for ≧CIN 2 is reached. So far, the addition of other methylation markers did not improve this sensitivity figure. We therefore reasoned that particularly in women with normal cytology the presence of a subset of ≧CIN 2 lesions is accompanied by absence of detectable genetic or epigenetic alterations, probably because these lesions represent earlier stages. For those cases, adding HPV type information may be of value to increase the sensitivity, given the previously mentioned increased risk of ≧CIN 2, particularly in women with normal cytology, conferred by HPV types HPV 16, 18, 31, 33 and/or 45. Indeed, by adding HPV type information better sensitivity figures were obtained. The combination of presence of methylation of CADM1 (Genbank ID NM_{—}014333), methylation of MAL (Genbank ID: NM_{—}022438) and/or presence of HPV 16 yielded a sensitivity for ≧CIN 2 in women with abnormal cytology of 95%. Furthermore, this marker combination revealed positively in 49% of hrHPV positive control women with normal cytology. Therefore, the use of this marker combination would refrain more than half of hrHPV positive women with normal cytology from unnecessary follow-up. A possible management algorithm would include referral for colposcopy of all hrHPV positive women with methylation of CADM1 (Genbank ID NM_{—}014333) and/or methylation of MAL (Genbank ID: NM_{—}022438). In this situation 83% of all women having ≧CIN 2 would be referred. The women positive for HPV 16 but without methylation could, on the other hand, be kept under surveillance given their increased risk of ≧CIN 2.

In another study using quantitative RT-PCR analysis on HPV-negative normal Pap smears, HPV-positive normal cervical scrapes and scrapes of women with abnormal cytology (moderate dyskaryosis or worse) we found that women who developed ≧CIN 2 within 18 months showed a significant increase in the ratio mRNA expression of DTX3L (Genbank ID: AK025135), PIK3R4 (Genbank ID: Y08991), ITGAV (Genbank ID: NM_{—}002210), ATP2C1 (Genbank ID: NM 014382) or SLC25A36 (Genbank ID: NM_{—}018155) versus MAL (Genbank ID: NM_{—}022438) as compared to women who did not develop high-grade CIN or worse (p=0.006). Hence, differential expression analysis at the mRNA or protein level of one or more of these genes may provide an alternative or additional marker panel in combination with hrHPV genotyping.

### 5. Marker Analysis of Self-Sampled Specimens

Similar results on methylation markers as outlined above were obtained from a study involving analysis of self-sampled cervico-vaginal samples. These self-samples were collected by women who, even after a second reminder, did not respond to the invitation for regular cervical screening, using either a Rovers® VibaBrush (Rovers Medical Devices, Oss, the Netherlands) or a Pantarhei® sampler (Pantarhei Devices, Zeist, The Netherlands). About one third of these women return self-sampled specimens to the lab. These samples are suitable for HPV PCR analysis (i.e. beta-globin PCR positive) and testing by hrHPV GP5+/6+-PCR yields at least as much ≧CIN 2 lesions in this population as found by regular screening in a matching population of responder women (Bais et al., Int J Cancer: 2007, 120:1505-1510). In fact, any kind of self-sampler, including a brush, swab, tampon, lavage or Pantarhei sampler, would suit to collect a cervical, vaginal or cervico-vaginal specimen as we found in a pilot study comparing various self-sampling devices for their performance.

Methylation analysis for CADM1 (Genbank ID NM_{—}014333; 1 promoter region) and MAL (Genbank ID: NM_{—}022438; 2 regions) by quantitative MSP was performed on self-samples of hrHPV positive women that later were diagnosed with ≧CIN 2 and hrHPV positive women without evidence of clinically meaningful disease in follow-up. Of the first group of women 69% revealed CADM1 and/or MAL methylation. Conversely, only about one third of hrHPV positive women without ≧CIN 2 showed methylation for either or both markers. After combining methylation data with hrHPV genotyping data it appeared that 84% of women diagnosed with ≧CIN 2 had CADM1 methylation, MAL methylation and/or presence of HPV 16, whereas of the number of marker positive hrHPV positive women without evidence of clinically meaningful disease in follow-up did not change markedly.

1. A method for cervical screening of women comprising
a) detection of high risk human papillomavirus (hrHPV) in cervical, cervico-vaginal or vaginal samples;
b) subjecting women positive in a) to marker analysis;
c) referring women positive in b) for colposcopy;
d) keeping women positive in a) for human papilloma virus (HPV) type 16, 18, 31, 33 and/or 45 and negative in b) under close surveillance;
e) referring women negative in a) or positive in a) for HPV types different from HPV 16, 18, 31, 33 and/or 45 and negative in b) to the next screening round.

2. A method for cervical screening of women comprising
a) detection of hrHPV in cervical, cervico-vaginal or vaginal samples;
b) subjecting women positive in a) to cytology;
c) referring women with abnormal cytology for colposcopy;
d) subjecting women with normal cytology to marker analysis;
e) referring women positive in d) for colposcopy;
f) keeping women with normal cytology positive in a) for HPV 16, 18, 31, 33, and/or 45 and negative in b) under close surveillance;
g) referring women negative in a) and those with normal cytology positive in a) for HPV types different from HPV 16, 18, 31, 33 and/or 45 but negative in d) to the next screening round.

3. A method for cervical screening of women comprising
a) marker analysis in cervical, cervico-vaginal or vaginal samples;
b) referring women positive in a) to colposcopy;
c) subjecting women negative in a) to hrHPV testing and typing;
d) keeping women positive in c) for HPV 16, 18, 31, 33 and/or 45 under close surveillance;
e) referring women negative in c) and those positive in c) for HPV types different from HPV 16, 18, 31, 33 and/or 45 to the next screening round.

4. A method for cervical screening of women comprising
a) marker analysis in cervical, cervico-vaginal or vaginal samples;
b) referring women positive in a) to colposcopy;
c) subjecting women negative in a) to cytology;
d) referring women with abnormal cytology to colposcopy;
e) referring women with normal cytology to the next screening round.

5. A method for cervical screening of women comprising
a) marker analysis in cervical, cervico-vaginal or vaginal samples;
b) referring women positive in a) to colposcopy;
c) subjecting women negative in a) to the next screening round.

6. Method according to claim 1, wherein said marker analysis involves testing with a marker panel consisting of expression markers and/or methylation markers.

7. Method according to claim 6, wherein said expression markers are selected from the group of ATP2C1 (Genbank ID: NM_{—}014382), SLC25A36 (Genbank ID: NM_{—}018155), DTX3L (Genbank ID: AK025135), CCDC14 (Genbank ID: AL122079), FLJ21291 (Genbank ID: AK024944), ITGAV (Genbank ID: NM_{—}002210), PIK3R4 (Genbank ID: Y08991, and MAL (Genbank ID: NM_{—}022438) and combinations thereof and wherein said methylation markers are selected from the group of MAL (Genbank ID: NM_{—}022438) and CADM1 (Genbank ID NM_{—}014333) and combinations thereof.

8. Method according to claim 7, wherein said methylation markers comprise a combination of MAL (Genbank ID: NM_{—}022438) and CADM1 (Genbank ID NM_{—}014333).

9. Method according to claim 6, wherein said methylation markers comprises MAL (Genbank ID: NM_{—}022438).

10. Method according to claim 1, wherein the cells, tissues or fluids are obtained from the cervical, cervico-vaginal of vaginal region by swab, brush, lavage, or by any other kind of (self-)sampler, including tampon and Pantarhei® sampler.

11. Method according to claim 1, wherein women testing negative from said marker analysis that are positive for HPV types different from HPV 16, 18, 31, 33 and/or 45 undergo follow-up testing after a time interval of at maximum 3 years to minimize the risk of interval high-grade lesions.

12. Method according to claim 4, wherein women negative in c) undergo HPV testing and typing and women positive for HPV types HPV 16, 18, 31, 33 and/or 45 will be kept under close surveillance and women positive for HPV types different from HPV 16, 18, 31, 33 and/or 45 will be referred to the next screening round or, alternatively, undergo follow-up testing after a time interval of at maximum 3 years to minimize the risk of interval high-grade lesions, while women negative for HPV will be referred to the next screening round.

13. Method according to claim 1, wherein women positive for HPV types 16, 18, 31, 33 and/or 45 are referred to colposcopy, whereas women positive for HPV types different from HPV 16, 18, 31, 33 and/or 45 are referred to the next screening round, or, alternatively, undergo follow-up testing after a time interval of at maximum 3 years.

14. Method to decrease the number of women subjected to follow-up testing after any type of primary testing by performing any of the methods according to claim 1.

15. Method to decrease the number of false positive and false negative results from cervical tissue screening by performing the method according to claim 1.

16. Method according to claim 1, wherein screening of individuals for any other HPV-associated mucosal (pre)cancer is indicated.

17. Method according to claim 2, wherein said marker analysis involves testing with a marker panel consisting of expression markers and/or methylation markers.

18. Method according to claim 17, wherein said expression markers are selected from the group of ATP2C1 (Genbank ID: NM_{—}014382), SLC25A36 (Genbank ID: NM_{—}018155), DTX3L (Genbank ID: AK025135), CCDC14 (Genbank ID: AL122079), FLJ21291 (Genbank ID: AK024944), ITGAV (Genbank ID: NM_{—}002210), PIK3R4 (Genbank ID: Y08991, and MAL (Genbank ID: NM_{—}022438) and combinations thereof and wherein said methylation markers are selected from the group of MAL (Genbank ID: NM_{—}022438) and CADM1 (Genbank ID NM_{—}014333) and combinations thereof.

19. Method according to claim 18, wherein said methylation markers comprise a combination of MAL (Genbank ID: NM_{—}022438) and CADM1 (Genbank ID NM_{—}014333).

20. Method according to claim 3, wherein said marker analysis involves testing with a marker panel consisting of expression markers and/or methylation markers.

21. Method according to claim 20, wherein said expression markers are selected from the group of ATP2C1 (Genbank ID: NM_{—}014382), SLC25A36 (Genbank ID: NM_{—}018155), DTX3L (Genbank ID: AK025135), CCDC14 (Genbank ID: AL122079), FLJ21291 (Genbank ID: AK024944), ITGAV (Genbank ID: NM_{—}002210), PIK3R4 (Genbank ID: Y08991, and MAL (Genbank ID: NM_{—}022438) and combinations thereof and wherein said methylation markers are selected from the group of MAL (Genbank ID: NM_{—}022438) and CADM1 (Genbank ID NM_{—}014333) and combinations thereof.

22. Method according to claim 21, wherein said methylation markers comprise a combination of MAL (Genbank ID: NM_{—}022438) and CADM1 (Genbank ID NM_{—}014333).

23. Method according to claim 4, wherein said marker analysis involves testing with a marker panel consisting of expression markers and/or methylation markers.

24. Method according to claim 23, wherein said expression markers are selected from the group of ATP2C1 (Genbank ID: NM_{—}014382), SLC25A36 (Genbank ID: NM_{—}018155), DTX3L (Genbank ID: AK025135), CCDC14 (Genbank ID: AL122079), FLJ21291 (Genbank ID: AK024944), ITGAV (Genbank ID: NM_{—}002210), PIK3R4 (Genbank ID: Y08991, and MAL (Genbank ID: NM_{—}022438) and combinations thereof and wherein said methylation markers are selected from the group of MAL (Genbank ID: NM_{—}022438) and CADM1 (Genbank ID NM_{—}014333) and combinations thereof.

25. Method according to claim 24, wherein said methylation markers comprise a combination of MAL (Genbank ID: NM_{—}022438) and CADM1 (Genbank ID NM_{—}014333).

26. Method according to claim 5, wherein said marker analysis involves testing with a marker panel consisting of expression markers and/or methylation markers.

27. Method according to claim 26, wherein said expression markers are selected from the group of ATP2C1 (Genbank ID: NM_{—}014382), SLC25A36 (Genbank ID: NM_{—}018155), DTX3L (Genbank ID: AK025135), CCDC14 (Genbank ID: AL122079), FLJ21291 (Genbank ID: AK024944), ITGAV (Genbank ID: NM_{—}002210), PIK3R4 (Genbank ID: Y08991, and MAL (Genbank ID: NM_{—}022438) and combinations thereof and wherein said methylation markers are selected from the group of MAL (Genbank ID: NM_{—}022438) and CADM1 (Genbank ID NM_{—}014333) and combinations thereof.

28. Method according to claim 27, wherein said methylation markers comprise a combination of MAL (Genbank ID: NM_{—}022438) and CADM1 (Genbank ID NM_{—}014333).
